(19) United States
(12) Patent Application Publication
(10) Pub. No.: US 2016/0022689 A1
(43) Pub. Date: Jan. 28, 2016

(54) Triazine compound for resisting coccidiosis in chickens

(71) Applicant: SHANGHAI VETERINARY RESEARCH INSTITUTE, CHINESE ACADEMY OF AGRICULTURAL SCIENCES, Shanghai (CN)

(72) Inventors: Lifang Zhang, Shanghai (CN); Feiqun Xue, Shanghai (CN); Chenzhong Fei, Shanghai (CN); Keyu Zhang, Shanghai (CN); Wenli Zheng, Shanghai (CN); Xiaoyang Wang, Shanghai (CN); Depeng Zhang, Shanghai (CN); Chao Fan, Shanghai (CN); Wenlong Xiao, Shanghai (CN); Mi Wang, Shanghai (CN); Tao Li, Shanghai (CN); Chunmei Wang, Shanghai (CN); Sui Xiao, Shanghai (CN)

(21) Appl. No.: 14/775,636

(22) PCT Filed: Nov. 6, 2014
(86) PCT No.: PCT/CN2014/090460

§ 371 Date: Sep. 11, 2015

(30) Foreign Application Priority Data
Nov. 8, 2013 (CN) 201310552795.2

(51) Int. Cl. A61K 31/53

(52) U.S. Cl. CPC A61K 31/53

(57) ABSTRACT

This invention is involved in the field of pharmaceutical technology. It is related to a triazine compound for resisting coccidiosis in chickens, its preparation method and application. This compound has the structure below. The invented drug has good efficacy, low toxicity and is suitable for treating animal coccidiosis

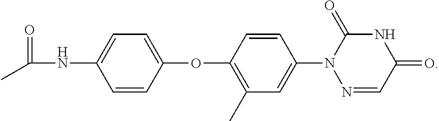 [SMILES: CC(=O)Nc1ccc(Oc2ccc(-n3ncc(=O)[nH]c3=O)cc2C)cc1]

### CROSS REFERENCE OF RELATED APPLICATION

This is a U.S. National Stage under 35 U.S.C 371 of the International Application PCT/CN2014/090460, filed Nov. 6, 2014, which claims priority under 35 U.S.C. 119(a-d) to CN 201310552795.2, filed Nov. 8, 2013.

### BACKGROUND OF THE PRESENT INVENTION

1. Field of Invention

This invention relates to a compound and its synthesis, and more particularly to a new triazine compound for resisting coccidiosis in chickens, its preparation method and applications.

2. Description of Related Arts

Chicken coccidiosis which is prevalent around the world is an important disease threatening intensive chicken farm. It is induced by Eimeria parasites in the enterocyte.

At present, there are two main methods for preventing chicken coccidiosis: vaccines and anticoccidial drugs. Although certain achievements have been made in the development and application of live coccidiosis vaccines, subunit vaccines and recombinant vaccines, the prevention and treatment of coccidiosis mainly depend on drugs. The development of anticoccidial drugs has significant effects on reducing the hazard of coccidiosis and the loss of chicken farm, and ensuring the development of poultry industry.

However, drug resistance caused by the long term usage of only one type of anticoccidial drug has been a veterinary clinical problem to be solved. Especially in chicken farms, the problem of drug resistance formed in coccidia is more widespread and more obvious. This drug resistance has become the biggest enemy of coccidiosis control in chicken farm. Studies showed that in Guangdong, China, 58% strains were sensitive to only one anticoccidial drug while 14% strains have lost sensitivity to all the drugs. Hence, drug resistance of coccidia is unfortunately widespread. It is less possible to choose the drugs with good treatment effects, which is the main reason why coccidiosis is hard to control in many areas. Due to the deficiency of new anticoccidial drugs, shuttle and rotation programs for drug administration have been clinically applied to avoid the further enhancement of drug resistance. This also restricts the development of poultry industry greatly. New generation of highly effective and low-toxicity anticoccidial drugs are required in veterinary clinics.

Triazine drugs, as currently the most active anticoccidial drugs, are widely applied around the world. The anticoccidial effect of triazine drugs needs further study. The most representative ones among triazine drugs are diclazuril and toltrazuril. Nevertheless, drug resistance has been generated after these drugs were used as feed additives for 20 years. So far, multiple chicken coccidia are resistant to diclazuril (Zhao qiping et al., Sensitivity of Eimeria species from two farms to Anticoccidial drugs in Anhui Province of China. Journal of Anhui Agricultural Sciences, 2010, 38 (21): 11142-11432) and toltrazuril (Wang et al., Observation of treatment effects of four common anticoccidial drugs on coccidiosis. Chinese Poultry, 31(24): 55-56), limiting the clinical application of these drugs.

Fortunately, the high anticoccidial activities of these compounds and the non-cross-resistance (e.g., diclazuril and toltrazuril have no cross-resistance) provide new opportunities for the development of new drugs.

In the Chinese patent application with the application No. of 200710040920.6, disclosed a new triazine compound for resisting coccidiosis in chickens, its preparation technique and applications. This compound is expressed with the structural formula (VI), based on which some pharmaceutically acceptable acid-addition or base-addition salts are formed: R₁ and R₂ represent one or several groups of hydrogen, halogen atom, alkyl, alkoxy, nitryl, and trifluoromethyl. The groups represented by R₁ and R₂ may be identical or not. R₃ represents COR₇, naphthenic base and heterocycle. R₄ represents hydrogen atom and alkyl group. The pharmaceutically acceptable acids include hydrochloric acid, sulfuric acid, hydrobromic acid, phosphoric acid, carbonic acid, formic acid, acetic acid, citric acid, lactic acid, fumaric acid, tartaric acid and gluconic acid. The pharmaceutically acceptable alkalines include sodium hydroxide, potassium hydroxide, triethylamine and tert-butylamine. These compounds have good inhibiting effect on animal coccidiosis.

Chinese patent application “Triazine Compound with Anticoccidial Activity, its Preparation Technique and Applications” (application No.: 200810202470.0) has published a type of anticoccidial triazine compound with the structural formula (VII), and listed the pharmaceutically acceptable acid-addition or base-addition salts formed: A represents oxygen or sulfur. R₁ and R₂ represent one or several groups of hydrogen, halogen atom, alkyl, naphthenic base, alkoxy, nitryl, trifluoromethyl, trichloromethyl, COR₅ and heterocycle; The groups represented by R₁ and R₂ may be same or not. R₃ represents hydrogen atom, alkyl group or naphthenic base. R₄ represents hydrogen atom, CO₂R₅ or CONHR₆. This compound has good inhibiting effect on animal coccidiosis.

Chinese utility patent for an invention “A Triazine Compound and its Application in Controlling Chicken Coccidiosis” (patent No.: 201110230812.1) has disclosed a compound with the structural formula (IV) and described its application in controlling chicken coccidiosis. Due to the existence of nitryl group in the chemical structure of this compound, there is a possibility of potential toxicity. Through acute oral toxicity test on rats, median lethal dose (LD₅₀) was calculated to be 768 mg/kg using improved Karber's method. The 95% confidence limit was 644-916 mg/kg.

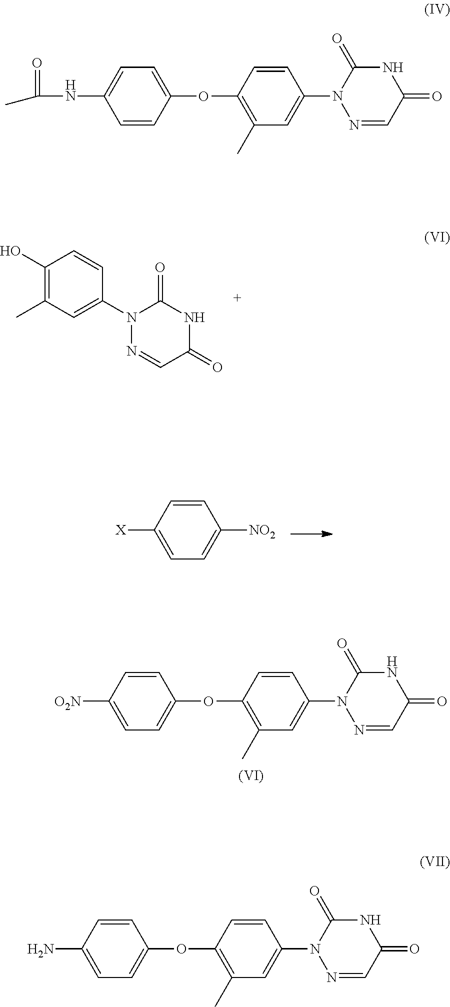

### SUMMARY OF THE PRESENT INVENTION

The objective of the present invention is to propose a new triazine compound for resisting coccidiosis in chickens, its preparation method and applications.

The present invention is accomplished in the following way: The structure of a triazine compound is shown below:

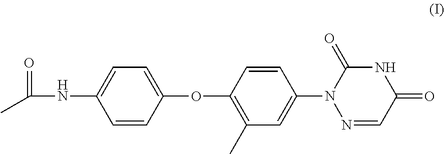 [SMILES: CC(=O)Nc1ccc(Oc2ccc(-n3ncc(=O)[nH]c3=O)cc2C)cc1]

The preparation of the above compound includes the steps below:

Step (A): performing williamson synthesis on a compound with a structural formula (II) or a sodium phenolate of the compound with the structural formula (II); and a compound with a structural formula (III) to be condensed into a compound with structural formula (IV); wherein a substituent x of the compound with the structural formula (III) is halogen, wherein in williamson synthesis, haloalkane is treated with alkoxide or phenoxide to obtain ether, which is a common method of ether bond synthesis, although this reaction has been discovered in 1852, it is still the best method for the synthesis of asymmetric ethers (Yu Lingchong (Ed.), Name Reactions in Organic Chemistry, Science Press, 1984: 345);

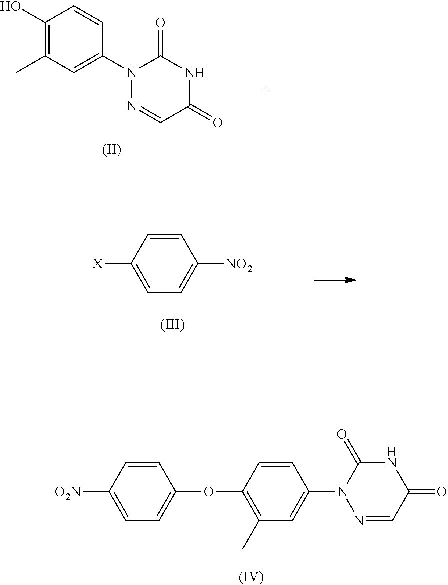

Step (B): reducing a nitro group in the compound with structural formula (IV) to obtain a compound with structural formula (V); and

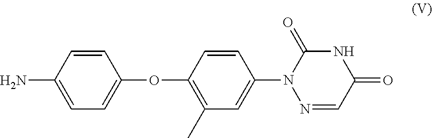 [SMILES: Cc1cc(-n2ncc(=O)[nH]c2=O)ccc1Oc1ccc(N)cc1]

Step (C): performing acylation on an amino group of the compound shown in the Eq (V) in the presence of acetyl chloride to obtain a compound shown in the Eq (I).

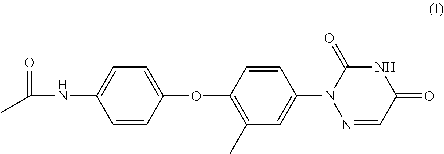 [SMILES: CC(=O)Nc1ccc(Oc2ccc(-n3ncc(=O)[nH]c3=O)cc2C)cc1]

In this invention, the compound with structural formula (II) can be prepared according to the method in U.S. Pat. No. 4,968,795A (see Line 25-66, Paragraph 22).

The compound with structural formula (I) as the active component or its pharmaceutical salt at an effective dose was mixed with pharmaceutical carrier to form an anticoccidial drug and then added into the feed. Its anticoccidial index (ACI) was 189 as drug concentration was 10 mg/kg.

So far, the compounds with structural formula (VI), (VII) and (IV) described above are not on the market. Their pharmacodynamic mechanisms and clinical applications are still being investigated. When investigating their pharmacodynamic mechanisms and drug metabolisms, multiple metabolic products with new structures were studied, such as the compounds with structural formula (I) and (V)which are metabolic products of the compound with structural formula (IV) in chicken body. The compound with structural formula (I) (code: AZL) showed difference from those with structural formula (VI), (VII) and (IV) and unexpected good anticoccidial effect. Its ACI reached 189 at the concentration of 10 mg/kg. Because AZL is the metabolic product of the compound with structural formula (IV), its toxicity further decreases. Through the acute oral toxicity test in rats, LD₅₀ of AZL compound was calculated to be 4743 mg/kg using improved Karber's method. The 95% confidence limit was 3754-5993 mg/kg.

Drug metabolism means that drug molecules are absorbed by the organism and then catalyzed by enzymes to generate a series of chemical reactions, which is also called biological transformation. During the long evolutionary process, the organism has developed a certain self-protection ability, and can chemically treat exogenous substances including drugs and toxic substances to discharge them easily to avoid damages. There are two main types of reactions related to the drug metabolism: 1. Functionalization reaction, also called phase I biological transformation reaction; 2. Binding reaction, also called phase II biological transformation reaction. Through functionalization reaction in vitro, the polar groups of metabolic products of drug molecules such as hydroxyl and amino groups can bind to the active endogenous small molecules such as glucuronic acid, sulfuric acid and amino acids via enzyme catalysis. This process is called binding reaction. Aromatic primary amines are mostly involved in the binding reaction due to acetylation in the metabolism. Amino groups which are formed via the reduction of aromatic nitro drugs may be bound through acetylation. Through N-terminal acetylation, the drugs will be mainly transformed into inactive or low-activity products. It means that this is an effective detoxication pathway (You Qidong (Ed.), Medicinal Chemistry (2nd Version), Chemical Industry Press, 2008, 59-66).

Unexpectedly, the experiments showed that the activity of AZL compound did not become lower and maintained good anticoccidial effect. When this compound was added into the feed at the concentration of 10 mg/kg, ACI was as high as 189. Because AZL is a new compound generated in the metabolic process, its toxicity further decreases. Through rats' acute oral toxicity test, LD₅₀ of AZL compound was calculated to be 4743 mg/kg using improved Larber's method. The 95% confidence limit was 3754-5993 mg/kg.

### DETAILED DESCRIPTION OF THE PREFERRED EMBODIMENT

The examples below are taken to introduce this invention. But there is the possibility that this invention can be achieved through other methods.

### Example 1

### Preparation of 2-{3-methyl-4-(4′-nitrophenoxy)phenyl}-1,2,4-triazine-3,5(2H,4H)-diketone {the compound with structural (IV)}

The compound with structural formula (II) (15 g, 0.068 mol), anhydrous sodium carbonate (7.9 g, 0.075 mol), parachloronitrobenzene (11.8 g, 0.075 mol) and dimethyl formamide (DMF, 150 ml) were added into the flask with four necks. The reaction lasted for 10 hours after heating to 120° C. After cooling to the room temperature, the reaction solution was transferred into 1000 ml water. Light yellow solids were separated out using 10% hydrochloric acid to reach the pH value of 3. The compound with structural formula (I) (16.2 g, 69.5%) was obtained via filtration, washing and drying.

Melting point (mp): 169-171.5° C. Electrospray ionization mass spectrometry (ESI-MS, m/z): 339.2(M-H)⁻; proton nuclear magnetic resonance (1H-NMR, CDCl₃): 2.25 (s, 3H), 7.00 (d, 2H), 7.09 (d, 1H), 7.44 (d, 1H), 7.50 (s, 1H), 7.60 (s, 1H), 8.22 (d, 2H), 9.69 (s, 1H).

### Example 2

### Preparation of 2-{3-methyl-4-(4′-aminophenoxy)phenyl}-1,2,4-triazine-3,5(2H,4H)⁻ diketone {the compound with structural formula (V)}

Reduced iron powder (9.8 g, 1.75 mol), water (50 ml), and ammonium chloride (3.75 g, 70 mmol) were added into the reaction flask and heated to 60° C. The ethyl acetate solution (100 mL) containing the compound with structural formula (IV) (11.9 g, 35 mmol) was added into reaction solution. Then the mixture was filtrated to separate organic phase after 6-7 hours of reflux reaction. The water phase was extracted with ethyl acetate. The organic phases were combined and washed to become neutral. Through drying with anhydrous sodium sulfate, filtration, and reduced pressure distillation to remove the solvent, earthy yellow solids {the compound with structural formula (V)} were obtained (8.68 g, recovery rate 80%).

ESI-MS (m/z): 309.2[M-H]⁻; 1H-NMR (400 MHz, DMSO-d6) δ: 2.27 (s, 3H), 6.60 (d, 2H), 6.68 (d, 1H), 6.75 (d, 2H), 7.19 (d, 1H), 7.34 (s, 1H), 7.59 (s, 1H).

### Example 3

### Preparation of 2-{3-methyl-4-(4′-acetamido phenoxy)phenyl}-1,2,4-triazine-3,5(2H,4H)⁻ diketone (the compound with structural formula (I))

The compound with structural formula (V) (15.0 g, 48 mmol) and triethylamine (9.1 g, 90 mmol) were dissolved in 60 ml DMF. When the temperature was lowered till below 5° C. via ice bath, acetyl chloride (6.0 g, 77 mmol) was slowly added into the solution. The reaction lasted for 30 min when the temperature was maintained below 5° C. via ice bath. Then as the temperature increased to the room temperature, the reaction lasted for 3 h. As the reaction was finished, an appropriate amount of water was added. Finally white solids (15.0 g, recovery rate 88.7%) were obtained through stirring, filtration, washing the filter cake to be neutral, methanol/water recrystallization and drying.

Mp: 215-218° C. ESI-MS(m/z): 351.1[M-H]⁻; 1H-NMR (400 MHz, DMSO-d6) δ: 2.03 (s, 3H), 2.24 (s, 3H), 6.86 (d, 1H), 6.94 (d, 2H), 7.28 (d, 1H), 7.41 (d, 1H), 7.58 (s, 1H), 7.61 (d, 2H), 9.94 (s, 1H), 12.31 (s, 1H).

### Example 4

### Preparation of AZL Premix

Firstly, 10000 g soybean meal (SBM) was added into 50 g AZL. They were well mixed and regarded as AZL premix. If needed, an appropriate amount of AZL premix would be added into chicken compound feed and well mixed.

### Example 5

Application of AZL in Preventing the Coccidiosis Induced by Eimeria tenella

1 Materials and Methods

1.1 Animals

One-day-old Pudong yellow feather cockerels

1.2 Drugs

1) AZL (99.10%), made by Shanghai Veterinary Research Institute, Chinese Academy of Agricultural Sciences (CAAS).

If needed, it is added into the chicken compound feed without other drugs and well mixed. Two AZL concentrations are set: 5 mg/kg and 10 mg/kg.

2) The compound with structural formula (V) (99.58%), made by Shanghai Veterinary Research Institute, CAAS.

If needed, it is added into the compound feed without other drugs and well mixed. Two drug concentrations are set: 5 mg/kg and 10 mg/kg.

3) The compound with structural formula (IV) (99.26%), made by Shanghai Veterinary Research Institute, CAAS.

If needed, it is added into the compound feed without other drugs and well mixed. Two drug concentrations are set: 5 mg/kg and 10 mg/kg.

4) Raw diclazuril material: If needed, it is added into the compound feed without other drugs and well mixed, with the diclazuril concentration of 1 mg/kg.

1.3 Experimental Methods

1.3.1 Grouping

After 90 one-day-old Pudong yellow feather cockerels were raised in the healthy animal room for 14 days. They were divided into 9 groups randomly, with 10 cockerels each group. One of these groups was the healthy control group. The AZL addition groups had two different treatments (5 mg/kg and 10 mg/kg). In the groups with the addition of compounds with structural formula (IV) and (V) respectively, two concentrations were set as AZL. The diclazuril addition group at the concentration of 1 mg/kg was taken as drug control group. Besides, an infection control group was also established.

1.3.2 Administration Method and Coccidial Infection

After grouping, the seven drug addition groups were fed with the feed containing specified drugs, while infection and healthy control groups were fed using pure feed without drugs. Each group drank freely. At the age of 15 day old, each cockerel in all the groups was infected with 80000 sporulated oocysts of Eimeria tenella, except for the healthy control group.

1.4 Clinical Observation

During the experimental period, food and water intake, mental state, illness and bloody stool were observed and recorded every day. At 5, 6 and 7 d after infection, the excrement in each group was collected to count the number of oocysts. The highest value was used for ACI calculation. During the experimental period, chicken death status was observed every day and the dead ones were autopsied. On the 8th day, chickens in all the groups were weighted and killed to observe and record weight gain and cecal lesion status.

1.5 Pharmacodynamic Assessment

1.5.1 Death rate: The percentage of the number of dead chicken because of infection to total number of experimental chicken.

1.5.2 Relative weight gain rate: The percentage of average weight gain of chickens in each infection group to that in the healthy control group

1.5.3 Cecal lesion score: It is scored by referring to the method by Johnson and Reid (Johnson J, Reid W M. Anticoccidial drugs: lesion scoring techniques in battery and floor-pan experiments with chickens.

Experimental Parasitology, 1970, 28 (1): 30-36). Cecal lesion score=average lesion score in a group×10.

1.5.4 Oocyst value: Cecal contents from all the chickens in each group were well mixed. The number of oocysts per gram (OPG) was calculated via Mc-Master's method. Ratio of oocyst number=(OPG in the negative control group or drug addition group÷OPG in the positive control group)×100% If the ratio is 0-1%, oocyst value is 0; if the ratio is 2%-25%, oocyst value is 5; if the ratio is 26%-50%, oocyst value is 10; if the ratio is 51%-75%, oocyst value is 20; if the ratio is 76%-100%, oocyst value is 40.

1.5.5 Anticoccidial index: ACI=(relative weight gain rate+survival rate)−(cecal lesion score+oocyst value)

1.5.6 Assessment criteria: When ACI is below 120, the drug is ineffective; when ACI ranges between 120 and 160, it is less effective; when ACI ranges between 160 and 180, it is moderately effective; when ACI is above 180, it is highly effective (Office of Veterinary Drug Evaluation Committee of the Ministry of Agriculture. Compilation of Technical Standards of Veterinary Drug Experiments China Agricultural Science and Technology Press, 2001, 22-23).

1.6 Results

The results showed that AZL had better anticoccidial effects at two different concentrations (Table 1).

TABLE 1
Experimental results of AZL application in preventing
the coccidiosis induced by Eimeria tenella

| | Drug | | Relative | | | |
|---|---|---|---|---|---|---|
| | concen- | | weight | | Cecal | |
| | tration | Survival | gain | Oocyst | lesion | |
| Groups | mg/kg | rate % | rate % | value | score | ACI |
| AZL | 5 | 100 | 90.8 | 5 | 5 | 180.8 |
| | 10 | 100 | 99.1 | 5 | 5 | 189.1 |
| Compound with | 5 | 100 | 92.2 | 5 | 5 | 182.2 |
| structural | 10 | 100 | 95.0 | 0 | 5 | 190.0 |
| formula IV | | | | | | |
| Compound with | 5 | 100 | 81.5 | 15 | 15 | 151.5 |
| structural | 10 | 100 | 83.1 | 15 | 10 | 158.1 |
| formula V | | | | | | |
| Diclazuril | 1 | 100 | 92.1 | 5 | 5 | 182.1 |
| Infection control | 0 | 70 | 65.7 | 40 | 28 | 67.7 |
| Healthy control | 0 | 100 | 100 | 0 | 0 | 200 |

### Example 6

### Acute Toxicity Test (LD₅₀) of AZL in Rats

1.1 Tested Drug

AZL (99.10%): made by Shanghai Veterinary Research Institute, CAAS

Drug solvent: 0.5% sodium carboxymethyl cellulose

Before administration, AZL was added into 0.5% sodium carboxymethyl cellulose to prepare suspensions at different concentrations.

1.2 Experimental animals: Specific pathogen-free (SPF) Sprague Dawley rats (50% female vs 50% male) were purchased from Shanghai Super-B&K laboratory animal Co., Ltd. (license number: SCXK (Hu): 2008-0016). Male and female rats were raised separately in the cages for more than 4 days before the experiments.

1.3 Acute Toxicity Experiment

Through repeated preliminary experiments, the interval of LD₀-LD₁₀₀ was determined According to this interval, groups were divided and the interval between groups was determined. Eighty rats (180-220 g) were selected and randomly divided into 8 groups. Each group had 10 rats, with the same number of female and male. Drug dosage in 7 groups were 2358, 3000, 3817, 4856, 6178, 7860, and 10000 mg/kg, respectively. The rats in these groups received oral gavage once. The remaining one group was chosen as negative control group which was drenched orally with drug solvent. After administration, this group was continuously observed for 14 days to calculate the death rate. Then LD₅₀ value and 95% confidence limit were calculated via improved Karber's method.

1.4 Results

Detailed experimental results are shown in Table 2. Through the calculation by improved Karber's method, LD₅₀ value was 4743 mg/kg and 95% confidence limit was 3754-5993 mg/kg.

TABLE 2
Acute toxicity experiment of AZL

| | Dosage | Logarithm of | Death rate |
|---|---|---|---|
| Groups | mg/kg | dosage | % |
| Drug group 1 | 2358 | 3.373 | 0 |
| Drug group 2 | 3000 | 3.477 | 30 |
| Drug group 3 | 3817 | 3.582 | 50 |
| Drug group 4 | 4856 | 3.686 | 50 |
| Drug group 5 | 6178 | 3.791 | 70 |
| Drug group 6 | 7860 | 3.895 | 70 |
| Drug group 7 | 10000 | 4 | 90 |
| Drug solvent | 0 | | 0 |
| group | | | |

What is claimed is:

1. A triazine compound for resisting coccidiosis in chickens, with following structure:

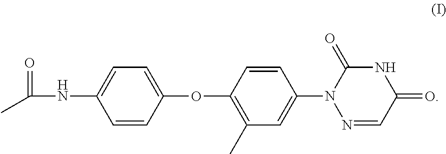 [SMILES: CC(=O)Nc1ccc(Oc2ccc(-n3ncc(=O)[nH]c3=O)cc2C)cc1]

2. An application of the compound mentioned in claim 1, with the peculiarity that this compound is used to prepare drugs for resisting coccidiosis in chickens.

3. A pharmaceutical composition for resisting coccidiosis in chickens, which is characterized in comprising: a significant amount of the triazine compound as recited in claim 1, a corresponding medicinal salt and a medicinal carrier thereof.

4. A preparation method for the compound as mentioned in claim 1, which is characterized in comprising following steps of:
step (A): performing Williamson synthesis on a compound shown in a structural formula (II) or a phenol sodium salt of the compound with the structural formula (II); and
a compound with the structural formula (III) to be condensed into a compound with a structural formula (IV); wherein a substituent X of the compound with the structural formula (III) is a halogen 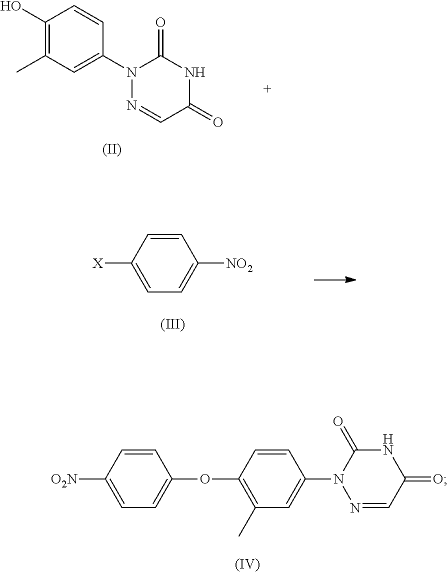
step (B): reducing a nitro group in the compound with the structural formula (IV) to obtain a compound with a structural formula (V); and 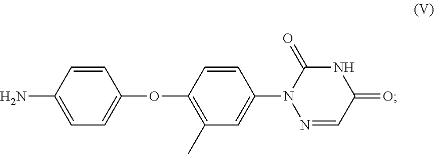 [SMILES: Cc1cc(-n2ncc(=O)[nH]c2=O)ccc1Oc1ccc(N)cc1]
step (C): performing acylation on an amino group of the compound with a structural formula (V) in the presence of acetyl chloride to obtain a compound with the structural formula (I) 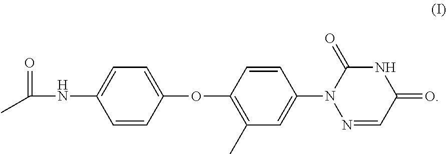 [SMILES: CC(=O)Nc1ccc(Oc2ccc(-n3ncc(=O)[nH]c3=O)cc2C)cc1]
